(19) United States
(12) Patent Application Publication
(10) Pub. No.: US 2023/0250449 A1
(43) Pub. Date: Aug. 10, 2023

(54) ENHANCED VIRAL TRANSDUCTION EFFICIENCY

(71) Applicant: MILLENNIUM PHARMACEUTICALS, INC., Cambridge, MA (US)

(72) Inventors: Nathan MOORE, Cambridge, MA (US); Fabio FACHIN, Cambridge, MA (US)

(21) Appl. No.: 17/995,211

(22) PCT Filed: Apr. 2, 2021
(86) PCT No.: PCT/US2021/025550

§ 371 Date: Sep. 30, 2022

Related U.S. Application Data
(60) Provisional application No. 63/004,979, filed on Apr. 3, 2020.

(51) Int. Cl. C12N 15/86; C12N 15/87; C12N 5/0783

(52) U.S. Cl. CPC C12N 15/86; C12N 15/87; C12N 5/0636; C12N 2510/00; C12N 2740/15043

(57) ABSTRACT

The present disclosure provides, among other things, a method of engineering genetically modified cells comprising, maintaining the cells in a collection chamber, contacting the cells with a fluid flow of a composition comprising viral or non-viral particles, thereby engineering genetically modified cells. The present disclosure also provides, among other things, a method of engineering genetically modified cells comprising, subjecting the cells to a centrifugal force, contacting the cells with a fluid flow of a composition comprising viral or non-viral particles, thereby engineering genetically modified cells.

### CROSS REFERENCE TO RELATED APPLICATIONS

This application claims priority to U.S. Provisional Application Ser. No. 63/004,979, filed Apr. 3, 2020, the disclosure of which is hereby incorporated by reference.

### BACKGROUND

Cell therapies take advantage of the natural transduction process, using virus particles modified for safety and functionality as a delivery vehicle (vector) for introducing therapeutic genes into a patient's cells. Viral vector transduction is currently the most frequently used method in cell therapy manufacturing for introducing therapeutic genetic material

Current manufacturing transduction processes are labor intensive and inefficient in the use of viral vectors, contributing to the high cost of manufacturing cell therapies and extending the time required to produce these therapies. Accordingly, there are significant limitations in the current state of the art manufacturing transduction processes.

### SUMMARY

The inventors have surprisingly conceived and devised an approach for vector-based transduction of cells that bypasses the limitations of current state-of-the-art methods. The disclosure provides flow-through, counterflow systems, for example counterflow centrifugation methods that allow for automated high efficiency cell transduction that can be applied to both lentivirus, retrovirus and other viral and non-viral particles.

In one aspect, a method of engineering genetically modified cells is provided comprising, maintaining the cells in a collection chamber, contacting the cells with a fluid flow of a composition comprising viral or non-viral particles, thereby engineering genetically modified cells.

In some embodiments, maintaining the cells in the collection chamber comprises subjecting the cells to a centrifugal force.

In one aspect, a method of engineering genetically modified cells is provided comprising, subjecting the cells to a centrifugal force, contacting the cells with a fluid flow of a composition comprising viral or non-viral particles, thereby engineering genetically modified cells.

In some embodiments, the centrifugal force is sufficient to maintain the cells in a cell bed.

In some embodiments, the direction of the fluid flow is counter to the direction of the centrifugal force.

In some embodiments, the fluid flow of the composition is sufficient to circulate the viral or non-viral particle without displacing the cells from the cell bed.

In some embodiments, recirculating the composition comprising the viral or non-viral particle.

In one aspect, a method of engineering genetically modified cells is provided comprising, subjecting the cells to a centrifugal force, contacting the cells with a fluid flow of viral or non-viral particles such that the direction of the fluid flow is counter to the direction of the centrifugal force, wherein the fluid flow is sufficient to maintain the cells in a cell bed, and circulating the viral or non-viral particles through the collection chamber, thereby engineering genetically modified cells.

In some embodiments, the collection chamber comprises an opening and an exit orifice opposite the opening to facilitate counter-flow and recirculation of the fluid composition.

In some embodiments, the centrifugal force is between about 20×g-3000×g. For example, in some embodiments, the centrifugal force is about 20×g, 50×g, 100×g, 200×g, 300×g, 400×g, 500×g, 600×g, 700×g, 800×g, 900×g, 1000×g, 1250×g, 1500×g, 1750×g, 2000×g, 2250×g, 2500×g, 2750×g, 3000×g.

In some embodiments, the fluid flow is at a constant flow rate.

In some embodiments, the constant flow rate is between 1 ml/min-150 ml/min. In some embodiments, the constant flow rate is between 1 ml/min-100 ml/min. For example, in some embodiments, the constant flow rate is about 1 ml/min, 5 ml/min, 10 ml/min, 15 ml/min, 20 ml/min, 25 ml/min, 30 ml/min, 35 ml/min, 40 ml/min, 45 ml/min, 50 ml/min, 55 ml/min, 60 ml/min, 65 ml/min, 70 ml/min, 75 ml/min, 80 ml/min, 85 ml/min, 90 ml/min, 95 ml/min, 100 ml/min, 105 ml/min, 110 ml/min, 115 ml/min, 120 ml/min, 125 ml/min, 130 ml/min, 135 ml/min, 140 ml/min, 145 ml/min or 150 ml/min.

In some embodiments, the fluid flow is at a pulse flow rate.

In some embodiments, the method comprises repeated cycles of a transduction or transfection phase and a viral or non-viral particle exchange phase.

In some embodiments, the transduction phase comprises, a centrifugal force of 0-50×g and a counter-flow flow rate of 0-10 ml/min. For example, in some embodiments, the centrifugal force is about 0×g, 2.5×g, 5.0×g, 10×g, 15×g, 20×g, 25×g, 30×g, 35×g, 40×g, 45×g, or 50×g. In some embodiments, the counter-flow rate is about 0 ml/min, 1 ml/min, 2 ml/min, 3 ml/min, 4 ml/min, 5 ml/min, 6 ml/min, 7 ml/min, 8 ml/min, 9 ml/min, or 10 ml/min.

In some embodiments, the virus exchange phase comprises a centrifugal force of 1500-3500×g and a counter-flow flow rate of 20-150 ml/min. In some embodiments, the virus exchange phase comprises a centrifugal force of 1500-3500×g and a counter-flow flow rate of 20-100 ml/min. For example, in some embodiments, the virus exchange phase comprises a centrifugal force of about 1500×g, 1750×g, 2000×g, 2250×g, 2500×g, 2750×g, 3000×g, 3250×g, or 3500×g. In some embodiments, the counter-flow flow rate is about 20 ml/min, 25 ml/min, 30 ml/min, 35 ml/min, 40 ml/min, 45 ml/min, 50 ml/min, 55 ml/min, 60 ml/min, 65 ml/min, 70 ml/min, 75 ml/min, 80 ml/min, 85 ml/min, 90 ml/min, 95 ml/min, 100 ml/ml, 105 ml/min, 110 ml/min, 115 ml/min, 120 ml/min, 125 ml/min, 130 ml/min, 135 ml/min, 140 ml/min, 145 ml/min or 150 ml/min.

In some embodiments, the viral or non-viral particle comprises a particle capable of introducing foreign nucleic acids into mammalian cells.

In some embodiments, the viral or non-viral particles are viral vector particles.

In some embodiments, the viral vector is derived from a lentivirus, retrovirus, adenovirus, adeno-associated virus, or a hybrid virus.

In some embodiments, the viral or non-viral particles are non-viral particles.

In some embodiments, the non-viral particles comprise liposomes, lipid particles, carbon, non-reactive metals, gelatin and/or polyamine nanospheres.

In some embodiments, the cells are B-cells, T cells, NK-cells, monocytes or progenitor cells.

In some embodiments, the method is performed in an automated closed system.

In some embodiments, the method is performed in a counter-flow centrifugation system.

In some embodiments, a population of cells is provided that is produced by a method described herein.

In some embodiments, a pharmaceutical composition is provided comprising cells produced by a method described herein.

In some embodiments, a method of manufacturing a population of cells is provided comprising engineering genetically modified cells by a method described herein.

Various aspects of the invention are described in detail in the following sections. The use of sections is not meant to limit the invention. Each section can apply to any aspect of the invention. In this application, the use of “or” means “and/or” unless stated otherwise. As used herein, the singular forms “a”, “an”, and “the” include both singular and plural referents unless the context clearly dictates otherwise.

### BRIEF DESCRIPTION OF THE DRAWINGS

FIG. 1 illustrates viral transduction in a standard static condition.

FIG. 2 illustrates a transduction chamber with volume V.

FIG. 3 illustrates a strategy of improving transduction rate with an increase in viral vector number.

FIG. 4 illustrates a strategy of improving transduction rate with an increase in the target cell number.

FIG. 5 illustrates a strategy of improving transduction rate with an increase in one or more of K, B_{R}, and E_{R}.

FIG. 6 illustrates a strategy of improving transduction rate by reducing the volume of the transduction chamber.

FIG. 7 illustrates the half-life of virus particles.

FIG. 8A illustrates a static system for viral transduction. FIG. 8B illustrates application of chemical enhancers in viral transduction. FIG. 8C illustrates application of spinoculation in viral transduction.

FIG. 9A illustrates a transport-driven viral transduction approach. FIG. 9B illustrates a physical confinement viral transduction approach. FIG. 9C illustrates an approach that combines the transport-driven and physical confinement approaches in viral transduction.

FIG. 10 illustrates a counter-flow centrifugation system.

FIG. 11 illustrates a transduction process in a counter-flow centrifugation system.

FIG. 12A illustrates a constant vector flow approach in viral transduction.

FIG. 12B illustrates a pulse vector flow approach in viral transduction.

FIG. 13 illustrates a vector MOI titration curve.

FIG. 14 illustrates an experimental design to test and compare the transduction rate achieved under three different conditions: a) an overnight static control condition, b) a 90 minutes static control condition, and c) a 90 minutes counter-flow centrifugation condition.

FIG. 15 illustrates the cell viability under three different conditions: a) an overnight static control condition, b) a 90 minutes static control condition, and c) a 90 minutes counter-flow centrifugation condition on Day 0 (Pre-transduction, Post-transduction), Day 1 and Day 5.

FIG. 16 illustrates the transduction rate achieved under three different conditions: a) an overnight static control condition, b) a 90 minutes static control condition, and c) a 90 minutes counter-flow centrifugation condition.

### DEFINITIONS

Adoptive Cell Therapy: As used herein, the term “adoptive cell therapy,” “adoptive cell transfer” or “ACT” refers to the transfer of cells into a patient in need thereof. The cells can be derived and propagated from the patient in need or could have been obtained from a non-patient donor. In some embodiments, the cell is an immune cell, such as a lymphocyte. Various cell types can be used for ACT such as, for example, a T-cells, CD8+ cells, CD4+ cells, NK-cells, delta-gamma T-cells, regulatory T-cells and peripheral blood mononuclear cells. In some embodiments, the cells are genetically modified to introduce a chimeric antigen receptor (CAR).

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Host cell or Target Cell: As used herein, the terms “host cell” or “target cell” includes cells that are not transfected, not infected and not transduced. In some embodiments, the terms “host cell” or “target cell” includes transfected, infected, or transduced with a recombinant vector or a polynucleotide of the invention. Host cells may include packaging cells, producer cells, and cells infected with viral vectors. In particular embodiments, host cells infected with viral vector of the invention are suitable for administering to a subject in need of therapy. In some embodiments, the target cell is a stem cell or progenitor cell. In certain embodiments, the target cell is a somatic cell, e.g., adult stem cell, progenitor cell, or differentiated cell. In preferred embodiments, the target cell is a hematopoietic cell, e.g., a hematopoietic stem or progenitor cell. In some embodiments, the target cell includes B-cells, T cells, NK-cells, monocytes or progenitor cells. In preferred embodiment, the target cell is T cells. In some embodiments, the target cell is a mammalian cell, an insect cell, bacterial cell, or fungal cell.

Functional equivalent or derivative: As used herein, the term “functional equivalent” or “functional derivative” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Non-viral particles: As used herein, the term “non-viral particles” includes non-viral carriers which are used for introducing nucleic acids into cells, for example, liposomes, lipid particles, carbon, non-reactive metals, gelatin and/or polyamine nanospheres.

Primary Cell: The term, “primary cell,” refers to cells that are directly isolated from a subject and which are subsequently propagated.

Polypeptide: The term, “polypeptide,” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of the disease, disorder, and/or condition.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Vector: As used herein, the term “vector” means the combination of any carrier and any foreign gene(s). The vector may include non-viral vectors, viral vectors, among others, and any combination thereof. For example, non-viral vectors may include but are not limited to liposomes, spheroplasts, red blood cell ghosts, colloidal metals, calcium phosphate, DEAE Dextran plasmids, among others, or a combination thereof. The viral vectors may include but are not limited to retroviral vectors, lentiviral vectors, pseudotype vectors, adenoviral vectors, adeno-associated viral vectors, hybrid virus, among others, and any combination thereof.

Transduction: As used herein, the term “transduction” means a process whereby foreign DNA is introduced into another cell via a viral vector. Various viral vectors are known in the art and include, for example, retroviral vectors, lentiviral vectors, pseudotype vectors, adenoviral vectors, adeno-associated viral vectors, among others, and any combination thereof.

Transfection: As used herein, the term “transfection” means a process of introducing nucleic acids into cells by non-viral methods. In some embodiments, the methods described herein are suitable for transfection of a cell of interest.

The recitation of numerical ranges by endpoints herein includes all numbers and fractions subsumed within that range (e.g. 1 to 5 includes 1, 1.5, 2, 2.75, 3, 3.9, 4 and 5). It is also to be understood that all numbers and fractions thereof are presumed to be modified by the term “about.”

Various aspects of the invention are described in detail in the following sections. The use of sections is not meant to limit the invention. Each section can apply to any aspect of the invention. In this application, the use of “or” means “and/or” unless stated otherwise. As used herein, the singular forms “a”, “an”, and “the” include both singular and plural referents unless the context clearly dictates otherwise.

### DETAILED DESCRIPTION

The inventors have surprisingly discovered a highly efficient method of transducing cells using flow-through, counterflow systems, such as for example counterflow centrifugation methods that allow for automated high efficiency cell transduction that can be applied to both lentivirus, retrovirus and other viral and non-viral particles. The methods described herein provide an approach towards vector-based transduction that bypasses the limitations of current state-of-the-art methods

### Viral Transduction in Cell Therapy

Current State of the Art

Transduction is the process through which viruses infect the cells of a host organism. Viruses naturally undergo the transduction process and have evolved to be very efficient at introducing genetic material into target cells. In order for transduction to occur, virus particles must come in physical contact with their target cells to first bind, enter, and finally integrate genetic material into the target cells. Binding occurs through specific protein-protein interactions, with the correct proteins needed on both the virus and target cell.

Cell therapies take advantage of the natural transduction process, using virus particles modified for safety and functionality as a delivery vehicle (vector) for introducing therapeutic genes into a patient's cells. Viral vector transduction is currently the most frequently used method in cell therapy manufacturing for introducing therapeutic genetic material.

Current industry approaches to viral transduction include static transduction systems, the use of chemical enhancers, and spinoculation. Each of these current industry approaches is further described below.

Viral transduction under static conditions is the most prevalent manner in which viral transductions are currently performed. Under standard static transduction methods, most transductions are performed in standard culture flasks or bags under static culture conditions. In this manner, viral vectors are suspended in media that can be about 100-1000s-fold deeper than the diameter of a single cell. Transduction using standard static methods face various problems that result in inefficient transduction of the cells. For example, using static methods results in the presence of small vector particles that remain in suspension and are unable to reach target cell. This is at least because large cells quickly sediment to the floor of culture vessels. The end result using the static culture methods for transduction is that only a small fraction of vector particles are capable of reaching cells through diffusion alone. As a result, transduction efficiency is low and the quantity of vectors needs to be high to achieve appreciable cellular transduction. This is because viral vector binding to a target cell is determined by receptor/ligand expression and physical contact. The transduction rate is thus proportional to the local concentration of virus for a given cell.

Another standard method for cellular transduction involves the use of chemical enhancers that in turn increase the binding rate of the vector to the cell. The use of methods that rely on chemical enhancers however is expensive and removal of the chemical enhancer creates an added barrier in the manufacturing process.

Another standard method for cellular transduction is the use of spinoculation. Spinoculation refers to centrifugal inoculation of cells. Spinoculation reduces the volume occupied by cells. This technique has been shown to have various negative aspects including, for example, damage to cells, difficulty in scaling up, and it is generally less effective for small vectors.

Cell Transduction Using Flow-Through, Counterflow Systems

The present disclosure provides methods that markedly increase the transduction efficiency of cells by increasing contact between vectors and target cells. In this manner, large quantities of cells are exposed to sufficient vector concentrations that allow efficient transduction of the cells. This results in reduced time for transducing cells while also minimizing vector waste. Therefore, the disclosure provides methods that reduce the total amounts of the vector used to achieve high transduction of the cells. Accordingly, in one aspect, the methods described herein achieve efficient cellular transduction at a reduced cost compared to conventional transduction systems. Additional benefits of the methods disclosed herein include an increased amount of transduced cells, less virus consumed during the transduction process, reduced process time, and reduced manufacturing costs. This in turn benefits patients at least because the methods allow for faster processing time, and the creation of a more potent therapeutic.

The methods described herein use fluidic flow to achieve efficient cellular transduction. The use of fluidic flow reduces diffusion lengths and prevents diffusion, each of which contribute to increased viral transduction efficiency.

In some aspects, a method of engineering genetically modified cells is provided comprising, maintaining the cells in a collection chamber, contacting the cells with a fluid flow of a composition comprising viral or non-viral particles, thereby engineering genetically modified cells. In some embodiments, the collection chamber comprises an opening and an exit orifice opposite the opening to facilitate counter-flow and recirculation of the fluid composition.

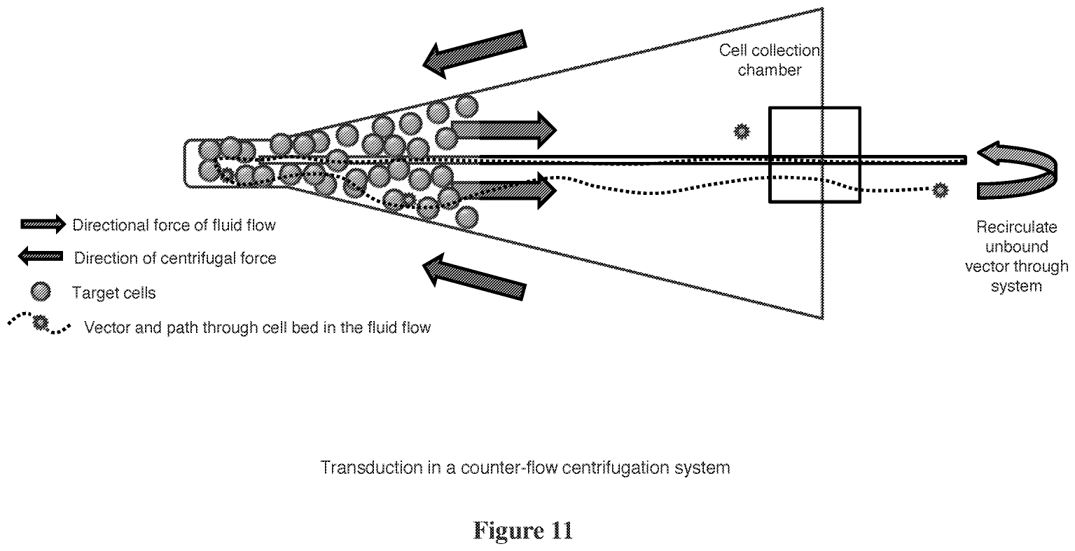

In some embodiments, the methods herein use counter-flow centrifugation systems. Counter-flow centrifugation systems are generally designed to concentrate and wash mammalian cells by balancing centrifugation with fluid flow to capture and contain cells. The counter-flow centrifugation systems do not pellet cells, but rather allow for continuous movement within the collection chamber. An exemplary counter-flow centrifugation system used for transduction of cells is illustrated in FIG. 11. In some embodiments, the counter-flow centrifugation system allows for about 5×10⁹ cells in a single batch. Accordingly, in some embodiments, the counter-flow centrifugation system allows for about 1×10⁹ cells, 2×10⁹ cells, 3×10⁹ cells, 4×10⁹ cells, 5×10⁹ cells, 6×10⁹ cells, or 7×10⁹ cells.

In some embodiments, the counter-flow centrifugation system allows for greater than 5×10⁹ cells in a single batch. In some embodiments, the counter-flow centrifugation system contains less than 5×10⁹ cells in a single batch.

In some embodiments, the methods described herein are automated to achieve multiple runs. In some embodiments, multiple runs accommodate greater than 5×10⁹ cells.

In some embodiments, the counter-flow centrifugation system has between about 5 to 10 mL harvest volume per round. Accordingly, in some embodiments, the counter-flow centrifugation system has about 3 mL, 4 mL, 5 mL, 6 mL, 7 mL, 8 mL, 9 mL, 10 mL, 11 mL, or 12 mL harvest volume per run.

In some embodiments, the counter-flow centrifugation system is run at a speed of between about 80 to 100 mL/min. Accordingly, in some embodiments, the counter-flow centrifugation system is run at a speed of about 70 mL/min, 75 mL/min mL/min, 80 mL/min, 85 mL/min, 90 mL/min, 95 mL/min, 100 mL/min, 105 mL/min, or 110 mL/min.

In some embodiments, the counter-flow centrifugation system flows between about 4 to 6 L/hr. Accordingly, in some embodiments, the counter-flow centrifugation system flows at about 3 L/hr, 3.5 L/hr, 4.0 L/hr, 4.5 L/hr, 5.0 L/hr, 6.0 L/hr, 6.5 L/hr, or 7.0 L/hr.

Without wishing to be bound by theory, the counter-flow centrifugation system of the methods described herein concentrates target cells into a high density cell bed using counter-flow centrifugation. Vector particles are too small to be affected by centrifugal force and are driven through the cell bed in the fluid flow where they bind and enter target cells. Recirculation of the vector particles through the system allows for multiple opportunities for vector particles to encounter and bind to target cells. In some embodiments, the counter-flow centrifugation system is automated in a closed system to perform transduction. The closed system allows for continuous circulation of the vector, thereby increasing contact of the vector with the cells. An exemplary schematic illustrating use of counter-flow centrifugation system in the transduction of cells is shown in FIG. 11.

In some embodiments, the centrifugal force in the counter-flow centrifugation system is between about 20×g-3000×g. For example, in some embodiments, the centrifugal force is about 20×g, 50×g, 100×g, 200×g, 300×g, 400×g, 500×g, 600×g, 700×g, 800×g, 900×g, 1000×g, 1250×g, 1500×g, 1750×g, 2000×g, 2250×g, 2500×g, 2750×g, 3000×g.

In some embodiments, the fluid flow is at a constant flow rate. In some embodiments, the constant flow rate is between 1 ml/min-150 ml/min. In some embodiments, the constant flow rate is between 1 ml/min-100 ml/min. In some embodiment, the constant flow rate is between 1 ml/min-10 ml/min. For example, in some embodiments, the constant flow rate is about 1 ml/min, 5 ml/min, 10 ml/min, 15 ml/min, 20 ml/min, 25 ml/min, 30 ml/min, 35 ml/min, 40 ml/min, 45 ml/min, 50 ml/min, 55 ml/min, 60 ml/min, 65 ml/min, 70 ml/min, 75 ml/min, 80 ml/min, 85 ml/min, 90 ml/min, 95 ml/min, 100 ml/min, 105 ml/min, 110 ml/min, 115 ml/min, 120 ml/min, 125 ml/min, 130 ml/min, 135 ml/min, 140 ml/min, 145 ml/min or 150 ml/min.

In some embodiments, the fluid flow is at a pulse flow rate. In some embodiments, the method comprises repeated cycles of a transduction or transfection phase and a viral or non-viral particle exchange phase.

In some embodiments, the transduction phase comprises, a centrifugal force of 0-50×g and a counter-flow flow rate of 0-10 ml/min. In some embodiment, the counter-flow flow rate is about 0-5 ml/min. For example, in some embodiments, the centrifugal force is about 0×g, 2.5×g, 5.0×g, 10×g, 15×g, 20×g, 25×g, 30×g, 35×g, 40×g, 45×g, or 50×g. In some embodiments, the counter-flow rate is about 0 ml/min, 1 ml/min, 2 ml/min, 3 ml/min, 4 ml/min, 5 ml/min, 6 ml/min, 7 ml/min, 8 ml/min, 9 ml/min, or 10 ml/min.

In some embodiments, the virus exchange phase comprises a centrifugal force of 1500-3500×g and a counter-flow flow rate of 20-150 ml/min. In some embodiments, the virus exchange phase comprises a centrifugal force of 1500-3500×g and a counter-flow flow rate of 20-100 ml/min. In some embodiment, the counter-flow rate is about 30-100 ml/min. For example, in some embodiments, the virus exchange phase comprises a centrifugal force of about 1500×g, 1750×g, 2000×g, 2250×g, 2500×g, 2750×g, 3000×g, 3250×g, or 3500×g. In some embodiments, the counter-flow flow rate is about 20 ml/min, 25 ml/min, 30 ml/min, 35 ml/min, 40 ml/min, 45 ml/min, 50 ml/min, 55 ml/min, 60 ml/min, 65 ml/min, 70 ml/min, 75 ml/min, 80 ml/min, 85 ml/min, 90 ml/min, 95 ml/min, 100 ml/ml, 105 ml/min, 110 ml/min, 115 ml/min, 120 ml/min, 125 ml/min, 130 ml/min, 135 ml/min, 140 ml/min, 145 ml/min or 150 ml/min.

In some embodiments, a constant vector flow approach is used with the counter-flow centrifugation system. Using a constant vector flow approach entails constant circulation of low flow vector throughout the transduction period. The flow is slow enough to allow vector particles to bind to cells. The vector is circulated to allow multiple chances for the vector to bind to the cells.

In some embodiments, a pulse vector flow approach is used with the counter-flow centrifugation system. Using a pulse vector flow approach entails cycles of long low/no flow periods followed by short bursts of high flow to replace vector within the collection chamber. Furthermore, low/no flow is long enough to allow vector to efficiently bind and enter target cells. A high flow period replenishes the chamber with unbound vector. The vector is also circulated to avoid loss and allow multiple chances for vector and cells to bind.

In some embodiments, the target cells are maintained in a collection chamber, and the target cells are contacted with a fluid flow of a composition comprising viral or non-viral particles, thereby engineering genetically modified cells. Accordingly, in some embodiments, the target cells are contacted with a viral particle. In some embodiments, the target cells are contacted with a non-viral particle.

Various kinds of viral particles are known in the art, and include, for example, retroviral vectors, lentiviral vectors, pseudotype vectors, adenoviral vectors, adeno-associated viral vectors, hybrid virus, among others, and any combination thereof.

Various kinds of non-viral particles are known in the art. In some embodiments, non-viral particles are used to engineer genetically modified cells. Examples of non-viral particles include, for example, liposomes, lipid particles, carbon, non-reactive metals, gelatin and/or polyamine nanospheres. Additional examples of non-viral particles include for example spheroplasts, red blood cell ghosts, colloidal metals, calcium phosphate, DEAE Dextran plasmids, among others, or a combination thereof.

In some embodiments, the method of genetically engineering cells is performed via transduction.

In some embodiments, the method of genetically engineering cells is performed via transfection using a non-viral particle.

In some embodiments, the methods described herein allow for shortened time to achieve a transduction of target cells in comparison to standard transduction methodology, such as static transduction methods or spinoculation methods.

### Uses of Transduced Cells

The transduced cells using the methods described herein allows for using the transduced cells for any purpose that a transduced cell can have. The transduced cells retain high viability (e.g., greater than 70%, 75%, 80%, 85%, 90%, 95%, 98%, or 99%) and can be used for a variety of applications, such as for cell therapy purposes such as, for example, in adoptive cell therapy applications.

Adoptive Cell Therapy

The methods described herein can be used, among other things, to genetically engineer cells for use in various therapeutic methods, including for example for use in adoptive cell therapy applications.

Adoptive cell therapy (“ACT”) refers to an infusion into patients of autologous or allogeneic cells to treat disease. Various cell types can be used for ACT-based therapies, such as B-cells, T cells, NK-cells, monocytes or progenitor cells. The progenitor cells can be isolated directly from a patient or from a non-patient donor. The progenitor cells include, for example, adult stem cells and pluripotent cells such iPSCs derived from a patient or non-patient donor. In some embodiments, ACT uses genetically modified hematopoetic stem cell (“HSC”) transplantation.

Hematopoietic stem cell (“HSC”) transplantation, one category of ACT methods, involves the infusion of autologous or allogeneic stem cells to reestablish hematopoietic function in patients whose bone marrow or immune system is damaged or defective. It also allows the introduction of genetically modified HSCs, for example to treat congenital genetic diseases. In typical HSC transplantation, the HSCs are obtained from the bone marrow, peripheral blood or umbilical cord blood.

In some embodiments, cells obtained from the peripheral blood are genetically engineered for use in ACT methods. Peripheral blood is used for autologous transplantations because of high stem cell and progenitor cell content as compared to bone marrow or cord blood. Moreover, HSCs obtained from peripheral blood show faster engraftment following transplantation. Because HSCs in the peripheral blood are present at low concentrations, the donor is typically treated with a mobilizing agent, such as granulocyte colony stimulating factor (G-CSF) or granulocyte macrophage colony stimulating factor (GM-CSF), which affects adhesion of HSCs to the bone marrow environment and releases them into the peripheral blood.

In some embodiments, the methods described herein are used to genetically modify T cells for T cell immunotherapy-based ACT methods. T cell immunotherapy is another category of ACT methods and involves the infusion of autologous or allogeneic T lymphocytes that are selected and/or engineered ex vivo to target specific antigens, such as for example tumor-associated antigens. The T lymphocytes are typically obtained from the peripheral blood of the donor by leukapheresis. In some T cell immunotherapy methods, the T lymphocytes obtained from the donor, such as tumor infiltrating lymphocytes (“TIL”s), are expanded in culture and selected for antigen specificity without altering their native specificity. In other T cell immunotherapy methods, T lymphocytes obtained from the donor are engineered ex vivo, typically by transduction with viral expression vectors, to express chimeric antigen receptors (“CAR”s) of predetermined specificity. CARs typically include an extracellular domain, such as the binding domain from a scFv, that confers specificity for a desired antigen; a transmembrane domain; and one or more intracellular domains that trigger T-cell effector functions, such as the intracellular domain from CD3ζ or FcRγ, and, optionally, one or more co-stimulatory domains drawn, e.g., from CD28 and/or 4-1BB. In still other T cell immunotherapy methods, T lymphocytes obtained from the donor are engineered ex vivo, typically by transduction with viral expression vectors, to express T cell receptors (“TCR”s) that confer desired specificity for antigen presented in the context of specific HLA alleles.

In some embodiments, the methods described herein are used to genetically modify hematopoietic stem cell (HSCs). In some embodiments, the HSCs are subject to additional treatments to expand the population of HSCs or manipulated by recombinant methods described herein to introduce heterologous genes or additional functionality to the allogeneic HSCs prior to transplantation into the recipient subject. In certain embodiments, the additional treatment leads to maturation of the HSCs.

HSCs obtained from a donor, either autologous or allogeneic, can be subject to additional treatments prior to transplantation into a recipient subject. In some embodiments, the HSCs are treated to expand the population of HSCs, for example by culturing one or more HSCs in a suitable medium.

In some embodiments, the HSCs, either autologous or allogeneic, are manipulated by recombinant methods to introduce heterologous genes by the methods disclosed herein. Such genetic manipulations can be used to correct genetic defects, and/or introduce additional functionality to the HSCs prior to transplantation. In some embodiments, a functioning wild type gene is introduced into the HSC to correct a genetic defect, for example, congenital hematopoietic disorders (e.g., β-thalassemia, Fanconi anemia, hemophilia, sickle cell anemia, etc.); primary immunodeficiencies (e.g., adenosine deaminase deficiency, X-linked severe combined immunodeficiency, chronic granulomatous disease, Wiskott-Aldrich syndrome, Janus kinase 3 deficiency, purine nucleoside phosphorylase (PNP) deficiency, leukocyte adhesion deficiency type 1, etc.); and congenital metabolic diseases (e.g., mucopolysaccharidosis (MPS) types I, II, III, VII, Gaucher disease, X-linked adrenoleukodystrophy, etc.). In certain embodiments, the HSCs are subjected to gene manipulation by recombinase systems, such as genome editing using CRISPR/Cas9 system or Cre/Lox recombinases. For example, the recombinase systems can be used to ablate genes or correct gene defects. In various embodiments, other methods of altering the functionality of HSCs include, among others, introduction of antisense nucleic acids, ribozymes, and RNAi.

### EXAMPLES

Other features, objects, and advantages of the present invention are apparent in the examples that follow. It should be understood, however, that the examples, while indicating embodiments of the present invention, are given by way of illustration only, not limitation. Various changes and modifications within the scope of the invention will become apparent to those skilled in the art from the examples.

### Transduction Rate

Transduction rate of a vector such as a viral vector is governed by the ability of the vector to bind to a target cell. The binding of the vector to the target cell is determined by a) an expression of ligand/receptor on the target cell, and b) a physical contact between the vector and the target cell.

Expression of Ligand/Receptor on a Target Cell

The type of the receptor on the target cell that the vector binds depends on the viral pseudotype, and the expression of a receptor depends on the target cell type and state of the cell. For example, for T cells, its activation is required to express a VSVG receptor. Generally, more than 90% binding between the target cell and the viral vector occurs within 3-5 minutes upon their exposure to each other. Therefore, the transduction rate is proportional to the local concentration of the virus around a target cell. Once a vector binds to the target cell, its entry kinetics depends on the cell type. Some cells are permissive, and allow a viral vector to enter the cell quite quickly. For instance, a human immunodeficiency virus (HIV) enters T cells within few minutes. In contrast, a less permissive cell takes several minutes to hours to allow the vector to enter the cell. For instance, entry of a HIV vector to a hematopoietic stem cell takes much longer time.

Physical Contact Between a Vector and a Cell

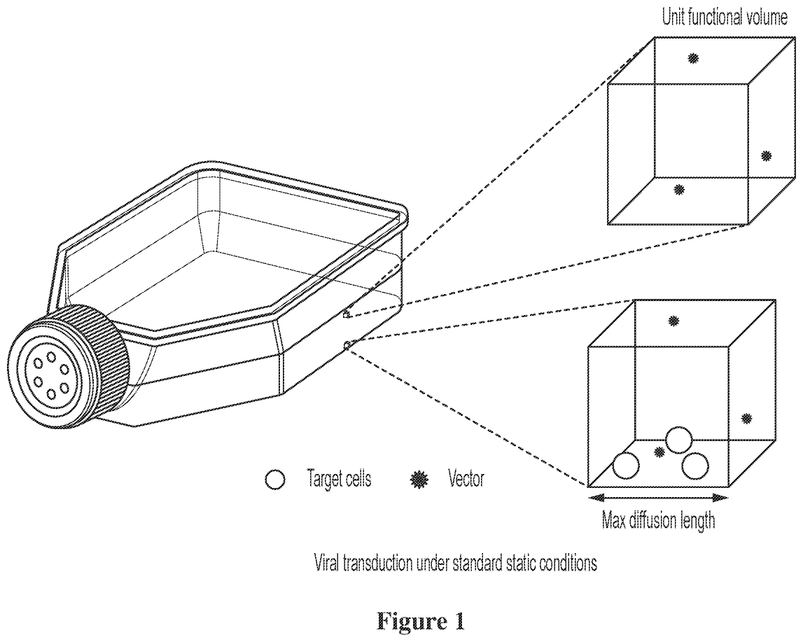

For a transduction to take place, a vector particle must come in contact or in proximity of the target cell. In most common transduction methods, transduction is carried out in standard culture flasks or bags under a standard static culture condition. In static conditions, viral vectors remain suspended in a culture media that is 100-1000 fold deeper than the diameter of a single cell. As most cells quickly sediment to the floor of the culture flask, only a fraction of vectors reach target cells through diffusion process. Thus, only a fraction of vectors come in contact with cells as illustrated in FIG. 1.

Transduction Rate Equation Applicable to Current Industrial Approaches

The transduction rate equation applicable to current industrial approaches is given below:

$${T}_{R} \approx {[ \frac{{W}_{\#}}{{T}_{\#}} ]}_{MOI} \frac{K * {B}_{R} * {E}_{R}}{V}$$

| Factor | Description |
|---|---|
| T_{R} | Transduction rate for a single cell |
| W_{#} | Viral vector number |
| T_{#} | Target cell number |
| K | Diffusion coefficient-rate of diffusion with a given time |
| B_{R} | Binding rate-controlled by expression of target cell |
| | receptors and vector pseudotype |
| E_{R} | Entry rate- specific for cell type |
| V | Volume of transduction chamber |

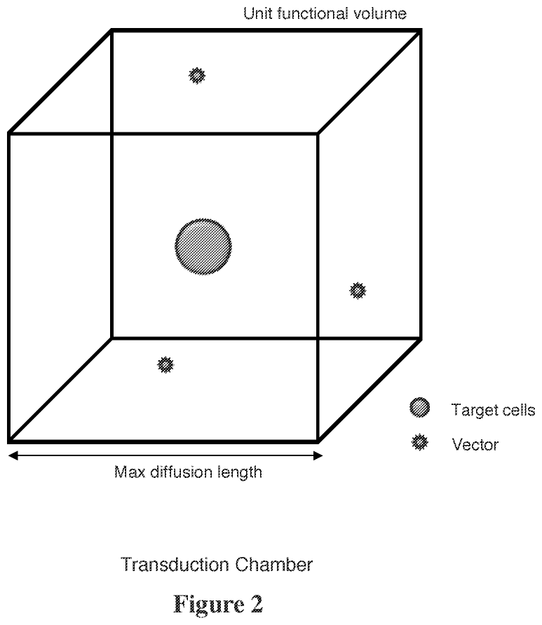

FIG. 2 illustrates a transduction chamber with a volume V. MOI stands for multiplicity of infection, i.e., the average number of virus particles infecting each cell during a transduction process.

### Different Strategies/Methods to Improve Transduction Rate

By Increasing the Number of Viral Vectors

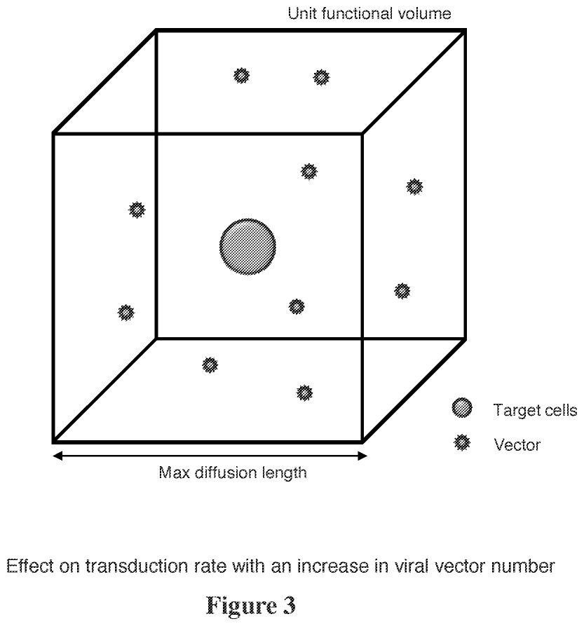

The transduction rate of a single cell can be improved by increasing the number of viral vectors per cell so that there is a greater likelihood a viral vector will contact and transduce each cell as illustrated in FIG. 3. However, this method involves an inefficient use of vectors/viruses, and therefore, it is an expensive method. Moreover, this method may not be feasible with low dilute viruses.

By Increasing Target Cell Numbers

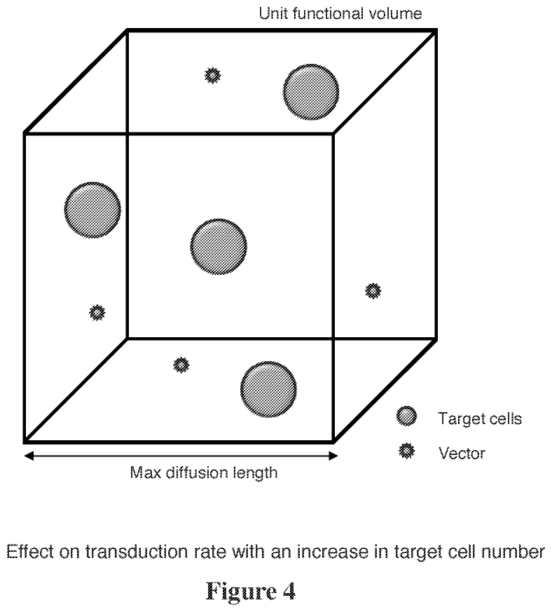

The transduction rate of a single cell can be improved by increasing the number of cells per viral vector so that more cells are available to be transduced by viruses as depicted in FIG. 4. However, this method require more cells, and thus lead to a reduction in the multiplicity of infection (MOI). Thus, the overall rate of transduced cells becomes lower.

By Increasing One or More of K, B_{R}, and E_{R}

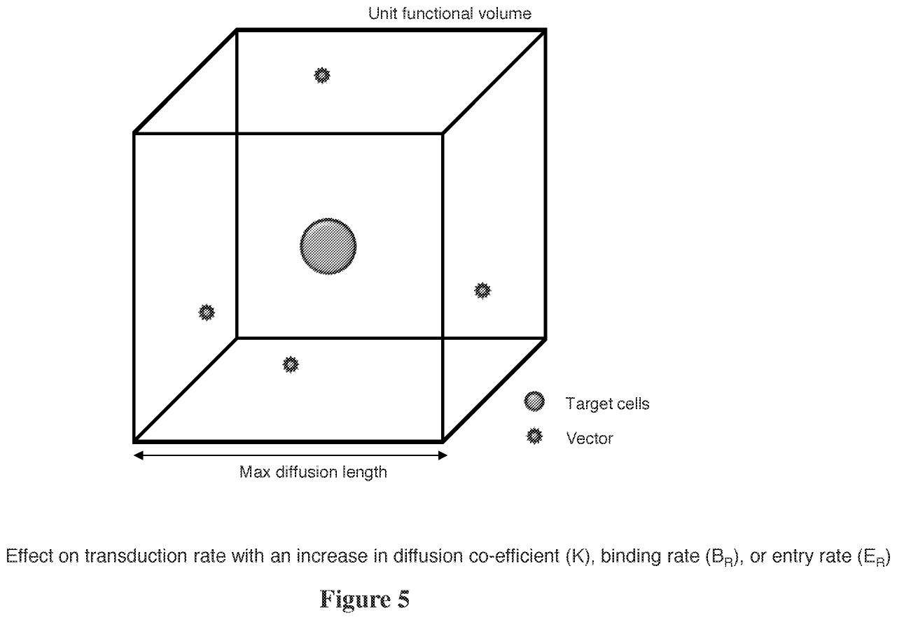

As K, diffusion coefficient, depends on the size of virus particle and the composition of fluid/media, it is quite difficult to vary. Similarly, B_{R}, and E_{R} depend on cell type and vector type, respectively, and therefore, they are also quite difficult to vary. FIG. 5 depicts this strategy. It will be difficult to change one or more of K, B_{R}, or E_{R} as it is difficult to tune the virus size or the target cell size.

By Reducing the Transduction Chamber Volume

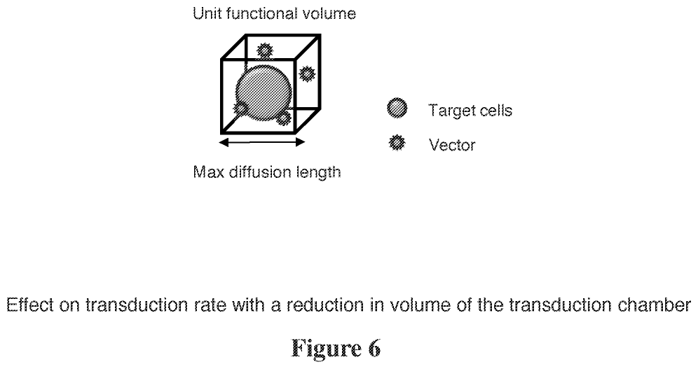

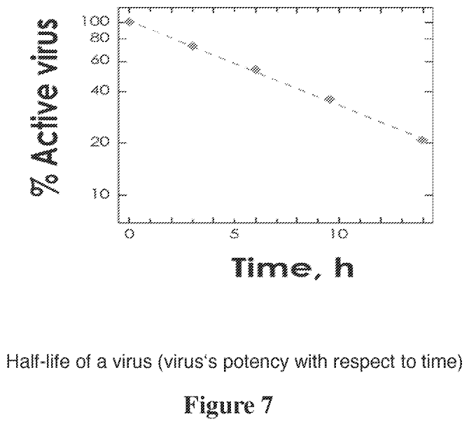

It is however feasible to reduce the volume of the transduction chamber. Smaller volume of the transduction chamber provides greater opportunity for virus and target cell interaction as depicted in FIG. 6. Thus, reducing the size of the transduction chamber decreases distances between cells and viral vector, and increases the likelihood that a viral vector will contact the target cell and consequent vector entry. This strategy does not require an increase in number of virus particles. Rather virus particles get higher chance to infect target cells during its half-life (i.e., 4-6 hours). FIG. 7 illustrates half-life of a virus (courtesy Tayi et. al. 2009).

### Current Industry Approaches to Viral Transduction

There are three current industry approaches to viral transduction: static systems, chemical enhancers, and spinoculation.

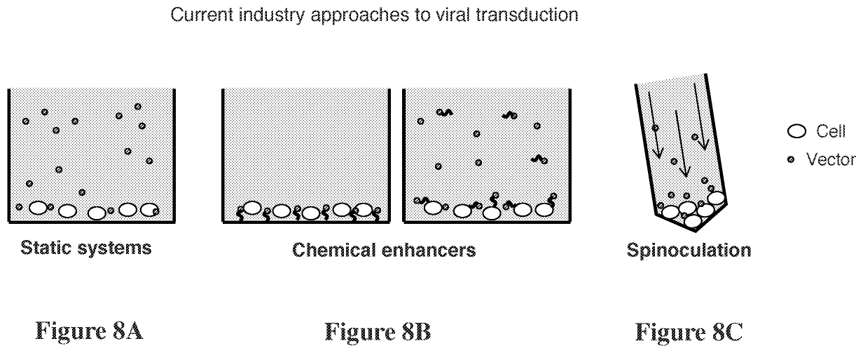

FIG. 8A depicts viral transduction in a static system. In a static system, the target cells are settled at the bottom of the container, for example, a culture flask, and the vector typically diffuses away from the target cells and remain in suspension. As a result, the transduction rate in a static system is low.

FIG. 8B depicts viral transduction in presence of chemical enhancers. Chemical enhancers are generally small molecules that are used to enhance the viral transduction process and increase target gene expression. Chemical enhancers temporarily increase the density of the a particular receptor on the target cell surface, including human cells, that are resistant to infection. Thus, chemical enhancers increase the B_{R}, the binding rate, in the transduction rate equation. Use of chemical enhancers can also be combined with a reduction in V, volume of the transduction chamber. However, use of chemical enhancers makes the transduction process expensive. Furthermore, the removal of chemical enhancers adds an additional problem in the manufacturing process.

FIG. 8C illustrates viral transduction using spinoculation process. Spinoculation (centrifugal inoculation or shell vial method) substantially improves the viral transduction rate. Although spinoculation process reduces V, volume of the reduction chamber, the full underlying mechanism of enhancement of viral transduction is so far unclear. Spinoculation process damages cells, and is less effective for small vectors. Spinoculation is also difficult to scale up in the manufacturing process.

### Application of Fluidic Flow Approaches to Viral Transduction

Fluidic flow prevents diffusion of vectors and reduces their diffusion length, and thereby improves transduction rate. Following fluidic flow approaches can be applied to improve viral transduction rate: transport-driven approach, physical confinement, a combination of transport and physical confinement approach, and counter-flow centrifugation.

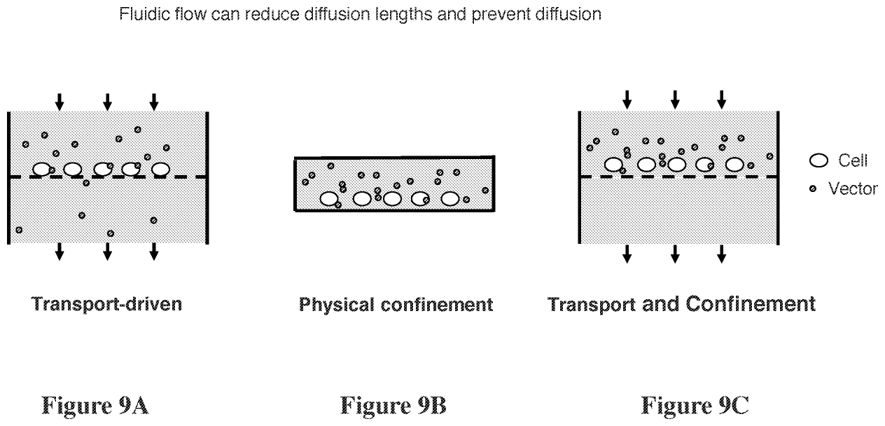

FIG. 9A illustrates a transport-driven viral transduction approach. Transport-driven approach uses a convective transport to deliver viruses to target cells. This approach reduces V, the volume of the transduction chamber for each cells, and also overpowers K, the diffusion co-efficient (diffusion rate of vector in a given time), and thereby improves the transduction rate. However, this approach requires a large amount of vectors.

FIG. 9B illustrates a physical confinement approach that applies fluidic flow. This approach confines cells and viruses in a microfluidic channel, and reduces V, volume of the transduction chamber for each cell. As a result, this approach improves the transduction rate. However, this approach requires pre-concentration of cells and vectors.

FIG. 9C illustrates an approach that combines the transport-driven and physical confinement approaches. This approach combines two concepts of co-concentration and convective transport in a microfluidic chamber. This approach reduces V, volume of the transduction chamber for each cell, and manipulates K, the diffusion co-efficient in the transduction equation, and thereby greatly improves the transduction rate.

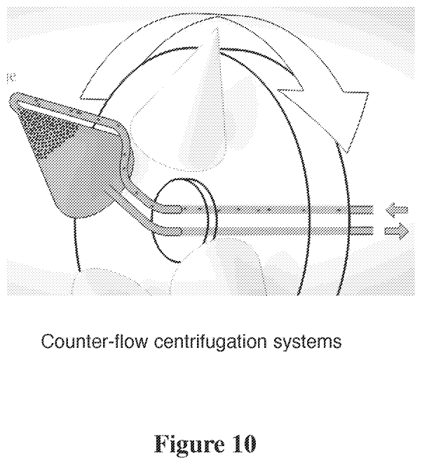

FIG. 10 illustrates counter-flow centrifugation approach. The counter-flow centrifugation approach is usually applied to concentrate and wash cells, for example mammalian cells, by balancing centrifugation with fluid flow to capture and contain cells. The counter-flow centrifugation approach does not result in pelleting of cells, rather facilitates continuous movement of cells within the collection/transduction chamber. In a typical counter-flow centrifugation system, up to 5 billion T cells can be run in a single batch. For more than 5 billion cells, a counter-flow centrifugation system can be automated for multi-round runs. The optimum run speed is typically about 80-100 mL/minute or 4-6 L/hour, and each run yields 5-10 mL of cell concentrate. Thermofisher's Rotea is an example of a counter-flow centrifugation system.

FIG. 11 illustrates the transduction process in a counter-flow centrifugation system. As can be seen, the counter-flow centrifugation system concentrates target cells into a high density cell bed. However, the vector particles/viruses are too small to be affected by the applied centrifugal force, and they pass through the cell bed in fluid flow and interact with target cells to infect them. The counter-flow centrifugation system allows recirculation of the unbound vectors through the cell bed, and thus vectors get multiple opportunities to encounter and bind target cells. As described previously, the counter-flow centrifugation system can be automated. This is a close system and allows recirculation.

### Transduction Rate Equation Applicable to Counter-Flow Centrifugation Approach

The transduction rate equation applicable to counter-flow configuration approach is given below:

$${T}_{R} \approx [ \frac{{W}_{\#}}{{T}_{\#}} ] \frac{{B}_{R} * {E}_{R} * {P}_{N}}{V}$$

| Factor | Description |
|---|---|
| T_{R} | Transduction Rate for a single cell |
| W_{#} | Viral Vector number |
| T_{#} | Target cell number |
| B_{R} | Binding rate- controlled by expression of target cell |
| | receptors and vector pseudotype |
| E_{R} | Entry rate- specific for cell type |
| V | Volume of transduction chamber |
| P_{N} | Number of vector passes through system |

The counter-flow centrifugation approach modifies the transduction rate equation that is applicable to current industrial transduction approaches. The transduction rate equation applicable to counter-flow configuration approach eliminates the dependency on diffusion (K) as diffusion is no longer required for driving vector towards the target cell. The transduction rate equation applicable to counter-flow configuration approach, however, introduces a new variable based on the number of times vector passes through the system, P_{N}. The counter-flow centrifugation approach also reduces V, the volume of the transduction chamber required for each cell.

### Vector Flow Approaches

In a transduction process, vectors can be introduced to the transduction chamber by one of the two approaches: constant vector flow and pulse vector flow.

Constant Vector Flow

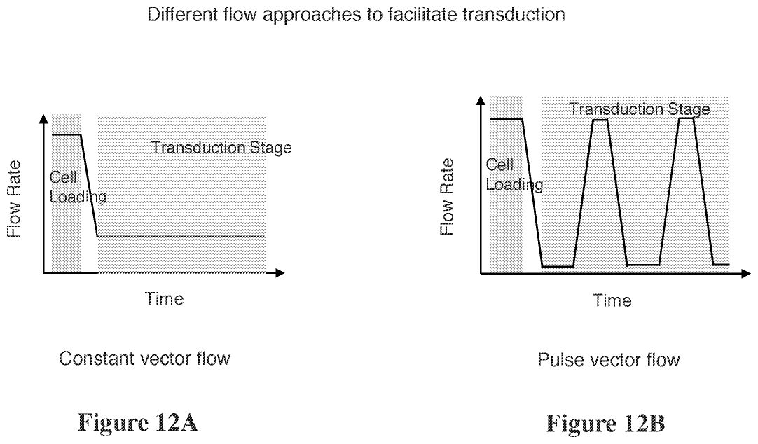

FIG. 12A illustrates a constant vector flow approach. Constant vector flow approach involves a constant circulation of low flow vector throughout transduction period. The flow is slow enough to allow virus particles to bind target cells. Vector is circulated to provide multiple chance for vectors to bind to cells.

Pulse Vector Flow

FIG. 12B illustrates a pulse vector flow approach. Pulse vector flow approach involves cycles of long low/no flow periods followed by short bursts of high flow to replace vectors within the collection chamber. The low/no flow periods are long enough to allow vector to efficiently bind and enter the target cells. The high flow period replenishes chamber with unbound vector. Vectors are circulated to allow vectors multiple chances to bind cells.

### Example 1. Initial Lentivirus Titration

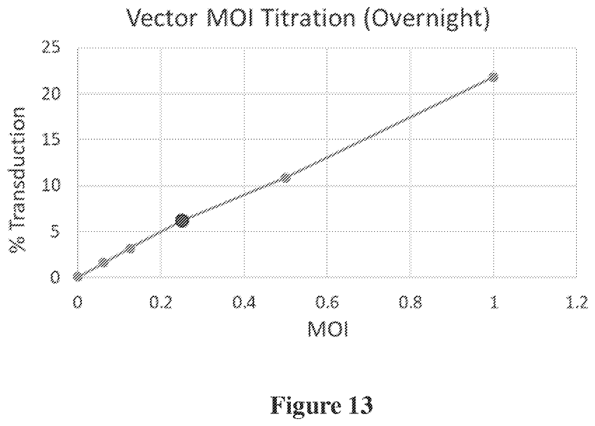

This example illustrates the initial lentivirus titration in T cells. In this example, a commercially available lentivirus with ZsGreen was used. Two-fold serial dilutions of the virus was prepared to determine optimum infectious range. Preactivated T cells plated in a standard 12-well plates were incubated with virus particles for 18 hours (overnight) under a static condition. Cells were expanded in 24 well plates for 5 days after transduction, and then the expanded cells were frozen. Flow cytometry was performed on thawed cells for cell viability and ZsGreen expression. A vector MOI titration curve was plotted, and is shown in FIG. 13.

MOI indicates the number of vector particles per cell used in the transduction. In this example, an MOI of 1 transduce about 22% of T cells.

### Example 2. Transduction Experiment Design

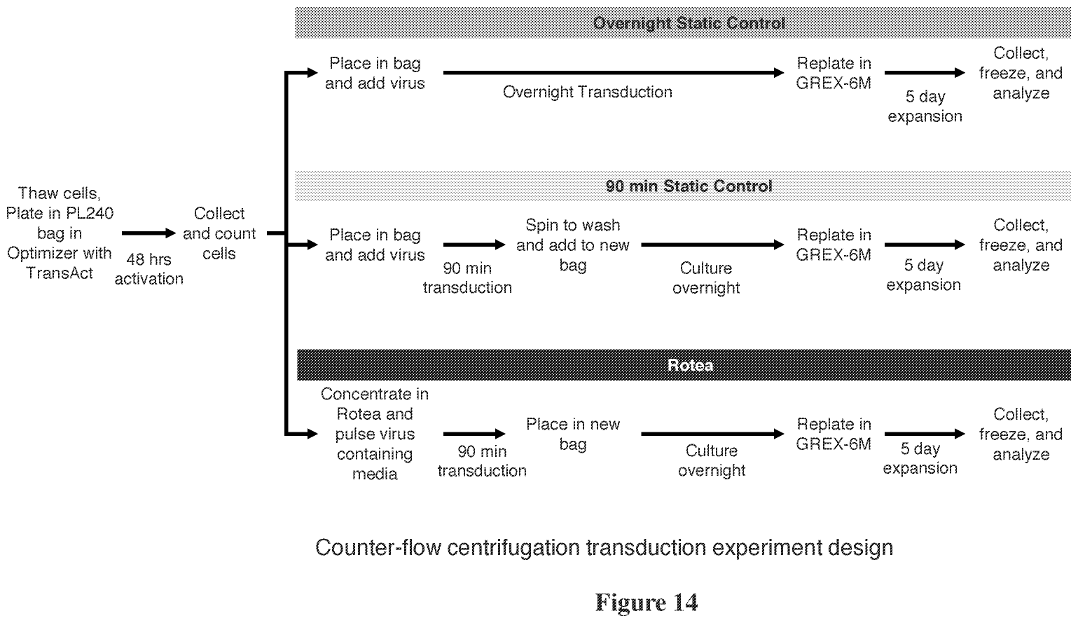

This example illustrates an experimental design to test and compare the transduction rate achieved under three different conditions: a) an overnight static control condition, b) a 90 minutes static control condition, and c) a 90 minutes counter-flow centrifugation condition. All these three different conditions are illustrated in FIG. 14.

In static control conditions, 7 million preactivated T cells at 1 million cells/mL concentration were taken in a PL07 bag. These preactivated cells were then transduced overnight or for 90 minutes with 1.75 IU virus (with MOI of 0.25).

In counter-flow centrifugation conditions, 100 million preactivated T cells were placed into a collection chamber, for example, in the Rotea collection chamber. The standard protocol was used to establish cell bed. The virus bag, for example, the Rotea virus bag, contained 30 mL media with 25 million infectious virus international unit (with effective MOI of 0.25). Virus was circulated through the cell bed under pulse using following conditions: transduction step: 3 min, 1 mL/min flow rate, and at 40×g; and virus exchange step: 10 sec, 30 mL/min flow rate, 3000×g; and circulation: 22 cycles in ˜90 minutes.

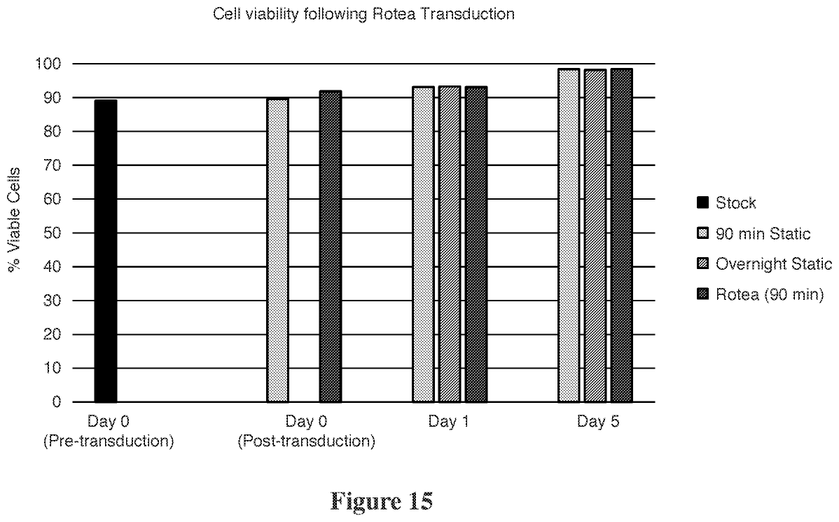

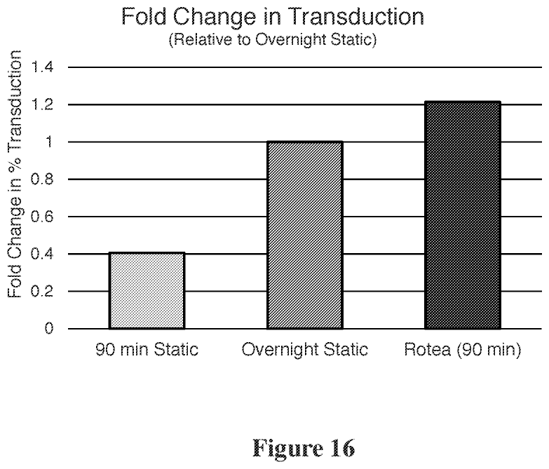

No difference was observed in cell viability immediately after transduction or after expansion among different conditions. Cell viability was determined using the NC-200 cell counter (Chemometec) and the result is shown in FIG. 15. Similarly, there was no significant difference in cell expansion among different conditions. The counter-flow centrifugation condition (performed using Rotea) allowed 100% recovery with respect to input volume compared to control conditions. As shown in FIG. 16, the transduction efficiency of the counter-flow centrifugation condition (using Rotea) in 90 minutes is equal to or higher than the transduction efficiency of overnight static condition.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of engineering genetically modified cells comprising,
maintaining the cells in a cell bed within a collection chamber,
contacting the cells with a fluid flow of a composition comprising viral or non-viral particles, and circulating the composition through the collection chamber, thereby engineering genetically modified cells.

2.-7. (canceled)

8. A method of engineering genetically modified cells comprising,
subjecting the cells to a centrifugal force,
contacting the cells with a fluid flow of a composition comprising viral or non-viral particles such that the direction of the fluid flow is counter to the direction of the centrifugal force, and wherein the fluid flow is sufficient to maintain the cells in a cell bed, and
circulating the viral or non-viral particles through the collection chamber, thereby engineering genetically modified cells.

9. The method of claim 8, wherein the collection chamber comprises an opening and an exit orifice opposite the opening to facilitate counter-flow and recirculation of the fluid composition.

10. The method of claim 8, wherein the centrifugal force is between about 20×g-3000×g.

11. The method of claim 8, wherein the fluid flow is at a constant flow rate.

12. The method of claim 11, wherein the constant flow rate is between 1 ml/min-100 ml/min.

13. The method of claim 8, wherein the fluid flow is at a pulse flow rate.

14. The method of claim 8, comprising repeated cycles of a transduction or transfection phase and a viral or non-viral particle exchange phase.

15. The method of claim 14, wherein the transduction phase comprises, a centrifugal force of 0-50×g and a counter-flow flow rate of 0-10 ml/min.

16. The method of claim 14, wherein the virus exchange phase comprises a centrifugal force of 1500-3500×g and a counter-flow flow rate of 20-100 ml/min.

17. The method of claim 8, wherein the viral or non-viral particle comprises a particle capable of introducing foreign nucleic acids into mammalian cells.

18. The method of claim 8, wherein the viral or non-viral particles are viral vector particles.

19. The method of claim 18, wherein the viral vector is derived from a lentivirus, retrovirus, adenovirus, adeno-associated virus, or a hybrid virus.

20. The method of claim 8, wherein the viral or non-viral particles are non-viral particles.

21. The method of claim 20, wherein the non-viral particles comprise liposomes, lipid particles, carbon, non-reactive metals, gelatin and/or polyamine nanospheres.

22. The method of claim 8, wherein the cells are B-cells, T cells, NK-cells, monocytes or progenitor cells.

23. The method of claim 8, wherein the method is performed in an automated closed system.

24. The method of claim 8, wherein the method is performed in a counter-flow centrifugation system.

25. A population of cells produced by a method of claim 8.

26. A pharmaceutical composition comprising cells produced by a method of claim 8.

27. (canceled)
